(19) United States
(12) Patent Application Publication
(10) Pub. No.: US 2023/0263991 A1
(43) Pub. Date: Aug. 24, 2023

(54) EXPANDABLE SHEATH FOR TRANSAXILLARY ACCESS

(71) Applicant: EDWARDS LIFESCIENCES CORPORATION, Irvine, CA (US)

(72) Inventors: Maxwell Harrison Fine, Costa Mesa, CA (US); Nasser William Saleh, Stockton, CA (US)

(21) Appl. No.: 18/142,179

(22) Filed: May 2, 2023

Related U.S. Application Data
(63) Continuation of application No. PCT/US2021/058077, filed on Nov. 4, 2021.
(60) Provisional application No. 63/109,433, filed on Nov. 4, 2020.

(51) Int. Cl. A61M 25/00; A61M 25/01

(52) U.S. Cl. CPC A61M 25/0023; A61M 25/01; A61M 2025/0024

(57) ABSTRACT

The expandable sheaths disclosed herein are adapted to be used in tortuous vessels, such as, but not limited to, the subclavian artery. The sheaths are expandable to facilitate passage of a medical device through a central lumen of the sheath. The sheaths can be used to deliver medical devices, such as implantable heart valves. The methods of delivering a medical device can include transforming a distal region of the expandable sheath to an at least partially bowed configuration and bending a central shaft of the expandable sheath around a bend in vasculature without kinking the sheath.

### RELATED APPLICATIONS

This application is a continuation of International Application No. PCT/US2021/058077, filed Nov. 4, 2021, which claims the benefit of U.S. Provisional Application No. 63/109,433, filed Nov. 4, 2020. Each of the aforementioned applications is incorporated herein by reference in its entirety for all purposes.

### FIELD

This disclosure is related to medical devices for cardiac procedures, and more specifically to expandable sheath technology for transcatheter procedures.

### BACKGROUND

Endovascular delivery catheter assemblies are used to implant prosthetic devices, such as prosthetic valves, at locations inside the body that are not readily accessible by surgery or where access without invasive surgery is desirable. For example, aortic, mitral, tricuspid, and/or pulmonary prosthetic valves can be delivered to a treatment site using minimally invasive surgical techniques.

A delivery catheter assembly can include a sheath to provide access to the vasculature for safely introducing a delivery apparatus. Radially expandable sheaths are a recent development in the field and reduce trauma to the insertion site by eliminating the need for multiple dilators to progressively increase the diameter of the insertion site. Radially expandable sheaths also minimize trauma to the interior vasculature by enabling a smaller diameter sheath. The sheath need only be temporarily expanded when necessary to allow for the passage of the delivery system. A radially expandable sheath generally has a radially expandable, elongated sleeve that is inserted into the vasculature and is coupled to a housing that contains one or more sealing valves. The sheath allows a delivery apparatus to be placed in fluid communication with the vasculature with minimal blood loss. The sheath acts to maintain hemostasis and facilitates the delivery of interventional devices, as well as wire and catheter exchanges.

For transcatheter aortic valve replacement (TAVR), the femoral artery is the most commonly used access vessel for the sheath because of its minimal invasiveness. The vessel is often close to the skin for easy access and has a relatively straight and simple path from the incision to the aortic valve compared to other approaches. However, in some cases, femoral access may not be feasible due to factors such as excessively tortuous anatomy, substantial calcification, or the presence of previously implanted grafts or stents. In these cases, alternative paths to the aortic valve must be considered. For TAVR, the most widely used alternative access is the axillary artery. Transaxillary approach is used in a significant and growing number of patients, and is considered a second option to transfemoral access in many centers (Biasco, Luigi, et al. “Access Sites for TAVI: Patient Selection Criteria, Technical Aspects, and Outcomes”). However, conventional expandable sheaths are designed for use in femoral anatomy and are not as well suited for transaxillary access.

### SUMMARY

An expandable sheath for transaxillary access is disclosed herein. The sheath is expandable from a collapsed configuration to a radially expanded configuration to facilitate passage of a medical device through the central lumen of the sheath. The expandable sheath is adapted to the length of the transaxillary and subclavian arteries. It is designed to bend without kinking in the tortuous subclavian anatomy, and it includes a distal region that minimizes trauma to the fragile anatomy around the aortic arch. For example, the expandable sheath can include a central lumen and a longitudinal axis extending therethrough, a tubular layer including an inferior longitudinal region and a superior longitudinal region, and a longitudinally extending reinforcement coupled to the inferior longitudinal region. The reinforcement includes a central axis extending parallel to the longitudinal axis of the sheath. The reinforcement is biased to bend in an inferior direction under axial compression.

In another example, the expandable sheath for transaxillary access includes a central lumen and a longitudinal axis extending therethrough and a tubular layer including a central shaft and a distal region. The distal region of the tubular layer is transformable to a bowed configuration under axial compression. The bowed configuration has a larger diameter than the shaft of the tubular layer; and a distal tip, wherein the distal tip is formed of a lower durometer material than a material of the tubular layer.

Methods of fabricating the expandable sheath for transaxillary access are also disclosed herein. One example method includes forming a tubular layer to expand when an inner surface of the tubular layer is subjected to a radially outwardly directed force, biasing a reinforcement to bend to a greater degree in a first direction than in the second, opposite direction, and coupling the reinforcement to an inferior longitudinal region of the tubular layer to make the expandable sheath.

Another example method includes forming a tubular layer to expand when an inner surface of the tubular layer is subjected to a radially outwardly directed force, altering a distal region of the tubular layer to be transformable to a bowed configuration under axial compression, and coupling a distal tip to the distal region of the tubular layer to make the expandable sheath, the distal tip being formed of a lower durometer material than the distal region.

The expandable sheaths disclosed herein can be used to deliver medical devices, such as implantable heart valves. Methods of delivering a medical device to a procedure site are disclosed herein. The methods of delivering the medical device include inserting an expandable sheath into a vessel of a subject, moving the distal tip of the expandable sheath toward a bend in the vessel, exerting pressure on the distal tip of the expandable sheath via contact with the vessel wall at the bend, transforming a distal region of the expandable sheath to an at least partially bowed configuration, advancing the distal tip of the expandable sheath distally past the bend in the vessel, bending a central shaft of the expandable sheath to an angle up to 120 degrees as the central shaft moves through the bend in the vessel, advancing a medical device through a central lumen of the expandable sheath, locally expanding the expandable sheath at a selected longitudinal position via an outwardly directed radial force applied by the advancing medical device, advancing the medical device through the bent portion of the central shaft, locally collapsing the expandable sheath at the selected longitudinal position once the medical device has advanced to a more distal location, and delivering the medical device to the procedure site.

### DESCRIPTION OF DRAWINGS

The device is explained in even greater detail in the following drawings. The drawings are merely exemplary and certain features may be used singularly or in combination with other features. The drawings are not necessarily drawn to scale.

FIG. 1 shows a sheath and a delivery apparatus for delivering a prosthetic device.

FIG. 2 shows a schematic of the aortic arch and subclavian arteries.

FIG. 3 shows a sheath kinked within a vessel.

FIG. 4 show a sideview of an aspect of a sheath for transaxillary access.

FIG. 5 shows a perspective cross sectional view of the sheath aspect of FIG. 4. The cross section is taken along the line from FIG. 4 marked FIG. 5.

FIG. 6 shows a cross sectional view of the sheath aspect of FIGS. 4 and 5. The cross section is taken along the plane from FIG. 5 marked FIG. 6.

FIG. 7A shows a bottom view of a reinforcement. FIG. 7B shows a perspective view of the reinforcement.

FIG. 8 is a diagram indicating an example flex profile of a reinforcement.

FIG. 9 shows the distal region of an aspect of a sheath for transaxillary access.

FIG. 10 shows an aspect of an expandable sheath bent without kinking within a tortuous vessel.

### DETAILED DESCRIPTION

The following description of certain examples of the inventive concepts should not be used to limit the scope of the claims. Other examples, features, aspects, embodiments, and advantages will become apparent to those skilled in the art from the following description. As will be realized, the device and/or methods are capable of other different and obvious aspects, all without departing from the spirit of the inventive concepts. Accordingly, the drawings and descriptions should be regarded as illustrative in nature and not restrictive.

For purposes of this description, certain aspects, advantages, and novel features of the aspects of this disclosure are described herein. The described methods, systems, and apparatus should not be construed as limiting in any way. Instead, the present disclosure is directed toward all novel and nonobvious features and aspects of the various disclosed aspects, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Although the operations of exemplary aspects of the disclosed method may be described in a particular, sequential order for convenient presentation, it should be understood that disclosed aspects can encompass an order of operations other than the particular, sequential order disclosed. For example, operations described sequentially may in some cases be rearranged or performed concurrently. Further, descriptions and disclosures provided in association with one particular aspect or implementation are not limited to that aspect or implementation, and may be applied to any aspect or implementation disclosed. It will be understood that various changes and additional variations may be made and equivalents may be substituted for elements thereof without departing from the scope of the disclosure or the inventive concept thereof. Certain aspects and features of any given aspect may be translated to other aspects described herein. In addition, many modifications may be made to adapt a particular situation or device to the teachings of the disclosure without departing from the essential scope thereof. Therefore, it is intended that the invention not be limited to the particular implementations disclosed herein, but that the invention will include all implementations falling within the scope of the appended claims.

Features, integers, characteristics, compounds, chemical moieties, or groups described in conjunction with a particular aspect, embodiment or example of the invention are to be understood to be applicable to any other aspect, embodiment or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The invention is not restricted to the details of any foregoing aspects. The invention extends to any novel one, or any novel combination, of the features disclosed in this specification (including any accompanying claims, abstract, and drawings), or to any novel one, or any novel combination, of the steps of any method or process so disclosed.

Throughout this application, various publications and patent applications are referenced. The disclosures of these publications in their entireties are hereby incorporated by reference into this application in order to more fully describe the state of the art to which this disclosure pertains. However, it should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. The terms “about” and “approximately” are defined as being “close to” as understood by one of ordinary skill in the art. In one non-limiting aspect the terms are defined to be within 10%. In another non-limiting aspect, the terms are defined to be within 5%. In still another non-limiting aspect, the terms are defined to be within 1%.

“Optional” or “optionally” means that the subsequently described event or circumstance may or may not occur, and that the description includes instances where said event or circumstance occurs and instances where it does not.

The terms “coupled,” “connected,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

The terms “proximal” and “distal” as used herein refer to regions of the delivery assembly or the sheath. “Proximal” means a region closer to the practitioner during a procedure, while “distal” means a region farther from the practitioner during a procedure. Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

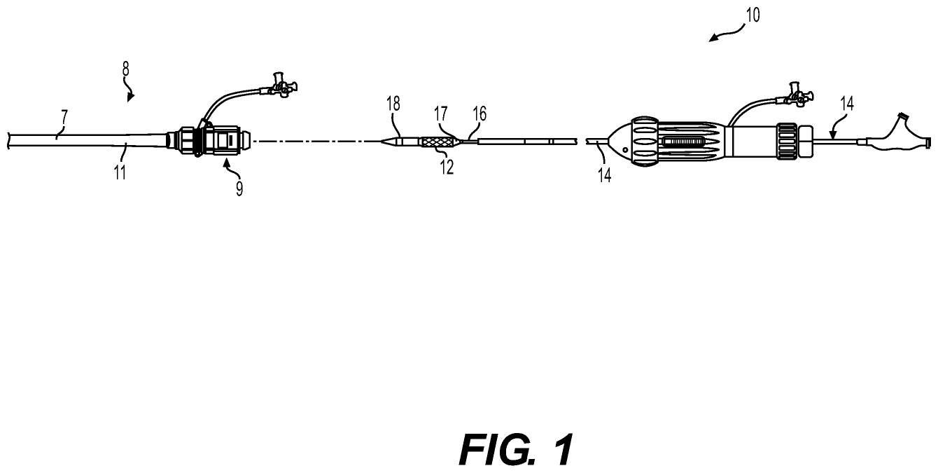

FIG. 1 illustrates a conventional sheath 8 and delivery apparatus 10 for delivering a medical device 12, such as a prosthetic heart valve, to a patient. Generally, distal end, central shaft 7, and proximal region 11 of sheath 8 are inserted into a vessel, passing through the skin of patient. A flexible, elongated introducer with a smoothly tapered distal end (not shown) is usually housed within the sheath 8, sticking out of the distal end of the sheath 8, to facilitate insertion. Sheath 8 can include a hemostasis valve within proximal hub 9, which stays outside the patient. Once the sheath 8 and proximal hub 9 are positioned, the introducer is removed. The distal region of delivery apparatus 10 is then inserted through proximal hub 9 of sheath 8. The delivery apparatus 10 can include a steerable guide catheter 14 (also referred to as a flex catheter), a balloon catheter 16 extending through the guide catheter 14, and a nose cone 18 at the distal end of balloon catheter 16. The guide catheter 14 and the balloon catheter 16 are adapted to slide longitudinally relative to each other to facilitate delivery and positioning of the device 12 at an implantation site in a patient's body. Once the medical device 12 is positioned, fluid advances through balloon catheter 16, expanding the balloon 17 and opening medical device 12 which is positioned over balloon 17. Once the medical device 12 is opened and positioned, the delivery apparatus 10 and the sheath 8 are removed from the patient.

Example expandable sheaths are disclosed in U.S. patent application Ser. No. 12/249,867, filed Oct. 10, 2008 (now U.S. Pat. No. 8,690,936), U.S. patent application Ser. No. 13/312,739, filed Dec. 6, 2011 (now U.S. Pat. No. 8,790,387), U.S. patent application Ser. No. 14/248,120 filed on Apr. 8, 2014 (now U.S. Pat. No. 9,301,840), U.S. patent application Ser. No. 14/324,894, filed Jul. 7, 2014 (now U.S. Pat. No. 9,301,841), U.S. patent application Ser. No. 15/057,953, filed Mar. 1, 2016 (now U.S. Pat. No. 9,987,134), U.S. patent application Ser. No. 15/997,587, filed Jun. 4, 2018, U.S. patent application Ser. No. 16/149,953, filed on Oct. 2, 2018 (now U.S. Pat. No. 10,524,905), U.S. patent application Ser. No. 16/149,956, filed on Oct. 2, 2018 (now U.S. Pat. No. 10,517,720), U.S. patent application Ser. No. 16/149,960, filed on Oct. 2, 2018 (now U.S. Pat. No. 10,524,906, U.S. patent application Ser. No. 16/149,969, filed on Oct. 2, 2018 (now U.S. Pat. No. 10,524,907), U.S. patent application Ser. No. 16/010,744, filed on Jun. 18, 2018 (no U.S. Pat. No. 10,639,152), U.S. patent application Ser. No. 14/880,111, filed on Oct. 9, 2015 (now U.S. Pat. No. 10,327,896), U.S. patent application Ser. No. 14/880,109, filed on Oct. 9, 2015, and U.S. patent application Ser. No. 15/914,748, filed on Mar. 7, 2018, the disclosures of which are herein incorporated by reference.

As noted above, conventional sheaths are typically designed for femoral access and do not perform as well when using a transaxillary approach. Three main issues arise. First, a sheath designed for femoral access is longer than necessary for the transaxillary approach. When it has been tracked as close to the aortic valve as possible, it will still stick out from the body. Conventional sheaths are designed such that, when the sheath is inserted over the femoral head, the tip of the sheath is past the aortic abdominal bifurcation in most patients. However, the distance from the transaxillary access to the native aortic annulus is shorter. When a large portion of the sheath is left outside the body, the sheath housing is unable to be sutured in place on the skin, and another person must manually hold the sheath in place, adding complication to the procedure. Furthermore, there is a greater percentage of the sheath shaft outside the body than for transfemoral access, which has implications for the hemostasis performance of the device. With the sheath shaft in the body, the blood that is inside the sheath is equal in pressure to the blood in the vessel surrounding the sheath. But when some of the sheath is outside the body, blood in the sheath shaft is at a higher pressure than the surrounding air, increasing the likelihood that blood will push on the walls of the sheath and cause deformation of the sheath walls and, in some cases, even leakage. Sheaths designed primarily for transfemoral access can include features near the proximal end to promote hemostasis in sections outside of the body. However, when these same sheaths are used for transaxillary access, more central sections of the sheath without hemostasis-promoting features are outside the body, increasing the chance of deformation and leakage.

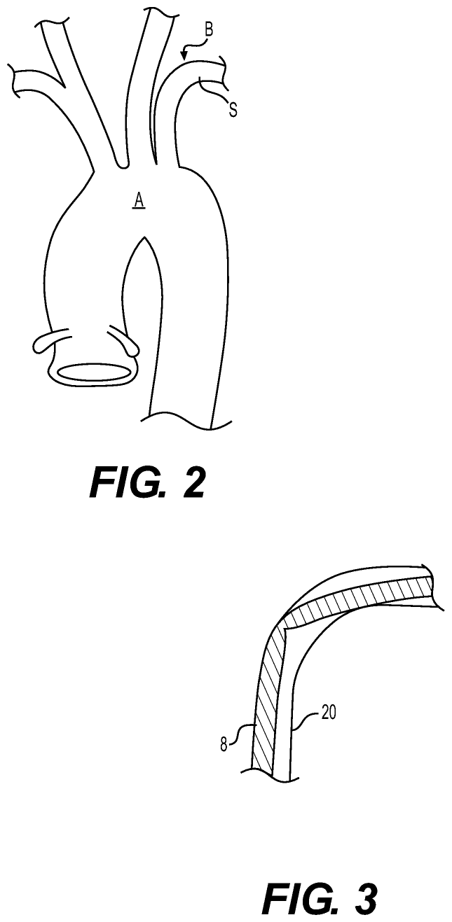

Secondly, the geometry of the transaxillary access is more tortuous than the transfemoral access geometry. See FIG. 2, for example. From the left side, there is an approximately 90-degree bend, B, that comes from the transition from the left subclavian artery S into the aortic arch A (the axillary artery becomes the subclavian artery approaching the heart). From the right side, although less commonly used, this bend can be even greater. By contrast, in the femoral anatomy, bends greater than 20 degrees are uncommon. Additionally, the path of the vessels for the axillary and subclavian anatomy are usually fairly rigid due to the presence of the surrounding clavicle and rib bones. In femoral anatomy, the artery has a greater freedom to move, as the surrounding bones are a greater distance from the vessel. To a certain degree, the sheath and introducer are able to straighten out femoral vessels, if especially tortuous, upon insertion. Thus, the path from incision to the aortic valve can almost always be expected to be more challenging on the sheath in axillary access than for femoral access.

Due to this more challenging geometry, conventional sheaths are more likely to kink in axillary access than in femoral access. A kink is a partial or full collapse of the inner lumen of the sheath, due to the deformation of the shaft on the inner curvature of a bend. FIG. 3 shows a conventional sheath 8 kinked within a bend of a tortuous vessel 20. The stiffness, kink resistance, and/or other kink-related specifications of conventional sheaths are chosen for the femoral approach, and are often not sufficient for the sheath to pass through the bend from the subclavian artery into the aortic arch. A kinked sheath can result in a failure to complete the procedure when the medical device 12 cannot be passed through the collapsed lumen of the sheath 8.

Third, the distal tips of conventional sheaths are also tailored for femoral access. During femoral access, the sheath and introducer are fully inserted into the anatomy. The introducer and sheath system together provide a smooth transition from the large outer diameter of the sheath, all the way down to the tip of the introducer, just bigger than the guidewire, making for an atraumatic access. Once in place, the introducer is removed. When fully inserted into the femoral artery, the distal tip of the sheath is past the abdominal aorta bifurcation, and the vessel is substantially large. The physician can reasonably expect that the distal tip of the sheath will not interact with the vessel walls in the abdominal aorta. However, when working within the transaxillary/subclavian anatomy, a physician must take great care not to allow the introducer to get too close to the aortic valve. Once the introducer is removed, the inflexible distal edge of the sheath is positioned very close to the native aortic valve. The distal edge can be pushed into the walls of the aortic arch and valve during the procedure, potentially causing damage.

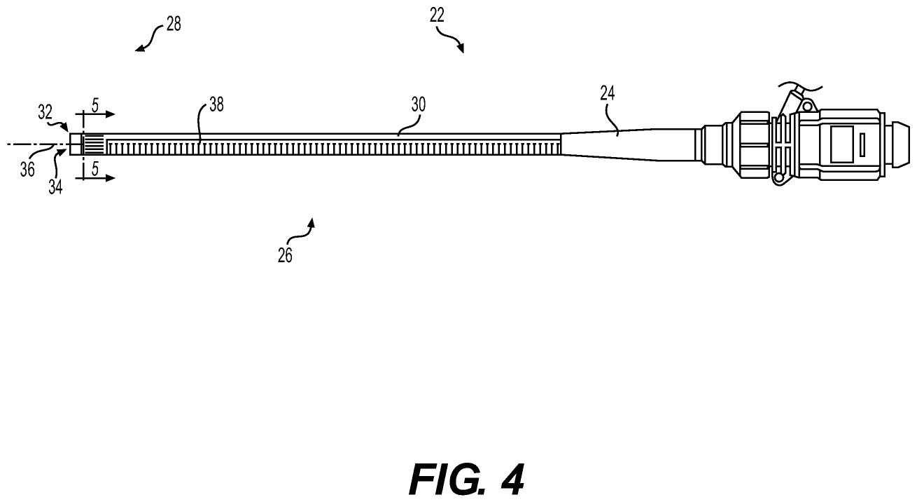

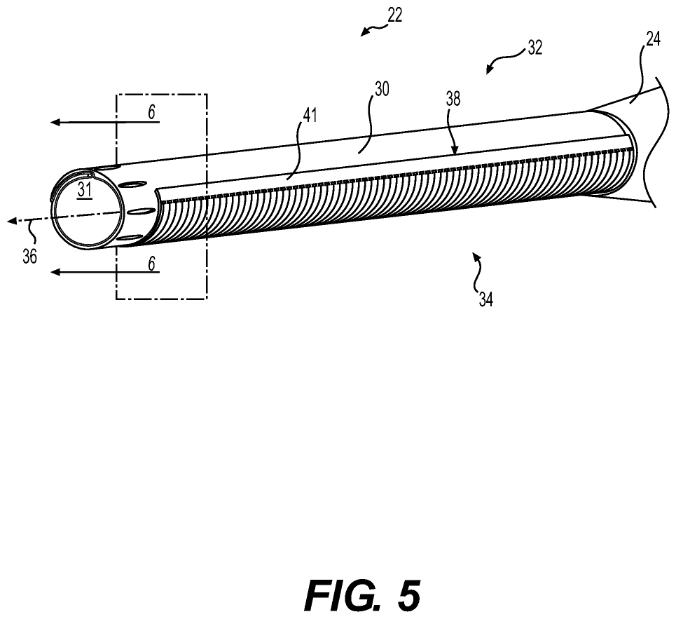

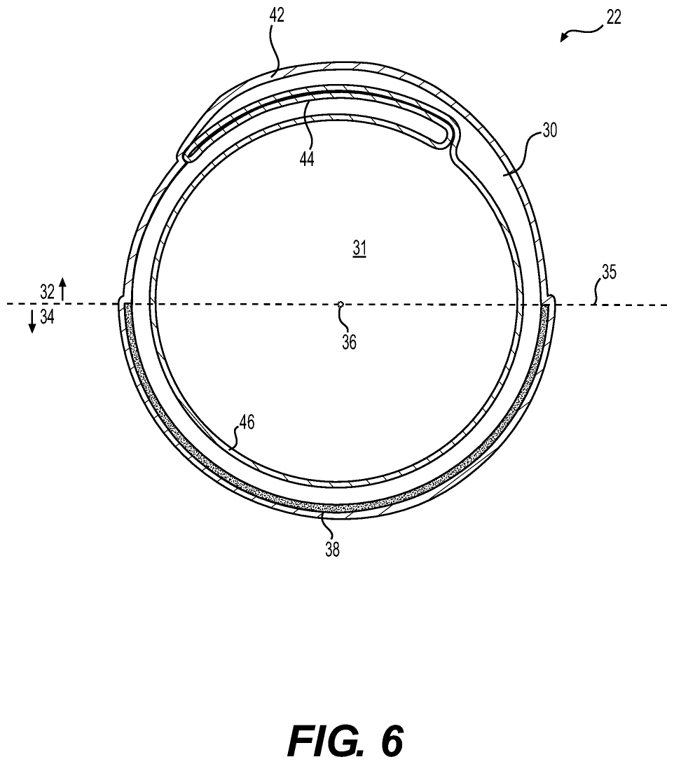

The sheaths and methods disclosed herein are tailored for the transaxillary anatomy to alleviate the issues described above. A side view of an example sheath for transaxillary access is shown in FIG. 4. Like conventional sheaths, sheath 22 includes a tapered proximal region 24, a central shaft 26, and a distal region 28. As shown in the perspective view of FIG. 5, the sheath has at least one tubular layer 30 that surrounds a central lumen 31 of sheath 22. The tubular layer 30 can be considered in terms of a superior longitudinal region 32 and an inferior longitudinal region 34, divided by a longitudinally extending plane 35 extending along the longitudinal axis 36 of the sheath 22 as shown in the transverse cross section of FIG. 6 and also in FIG. 4. The inferior longitudinal region 34 is intended to be positioned below the superior longitudinal region 32 during insertion into a subclavian artery. An outer jacket may cover the tubular layer of the sheath and is not shown in FIG. 5.

The sheath 22 is shorter than conventional sheaths. The length of the sheath 22, as measured between the proximal-most point of the proximal region 24 to the distal-most point of the distal region 28, can be from about 4 inches to about 10 inches (including about 4 inches, about 5 inches, about 6 inches, about 7 inches, about 8 inches, about 9 inches, and about 10 inches). In some aspects, the length of the sheath 22 is from about 5 inches to about 9 inches. For example, in some aspects, the sheath is about 7 inches long. For comparison, conventional expandable sheaths designed primarily for insertion into the femoral artery are about 12 inches long.

To address the issue of kinking, a reinforcement 38 is coupled to the external surface of the inferior longitudinal region 34. The reinforcement 38 is relatively rigid, having a higher durometer than the tubular layer 30. The reinforcement 38 prevents kinking of the sheath shaft, thereby mitigating risks associated with the high push force that would otherwise be required from the practitioner to advance the prosthetic device 12 through a collapsed lumen of the sheath. Because the sheath 22 must navigate the bend B in the subclavian artery S (see FIG. 2), it is biased to bend in an inferior direction when subjected to axial compression. As such, when the distal region 28 encounters the vascular wall at the bend B, it begins bending down toward the aortic arch A.

In some aspects, the reinforcement 38 can be coupled to the inferior longitudinal region 34 using adhesive placed, for example, along the interior surfaces of one or both of the left and right longitudinally extending spines 40, 41 of the reinforcement 38. In some aspects, the reinforcement might be coextruded with the tubular layer or heat bonded to the tubular layer. In some aspects, at proximal and distal ends of the reinforcement 38, additional layers of polymer can be placed over the reinforcement, extending longitudinally off one or both of the proximal and/or distal ends, and melted to the underlying tubular layer to reflow the additional layers of polymer to the tubular layer 30. The additional layers of polymer could be formed of the same or a different material than the tubular layer 30. In such an aspect, a central region of the reinforcement 38 would be free of additional layers of polymer because they could inhibit the ability of the reinforcement to bend. Some aspects can include, additionally or alternatively, an outer jacket 42 extending over the reinforcement and the tubular layer 30 (shown in FIG. 6). The outer jacket 42 can be formed of a highly elastic material that can radially compress a sheath 22 back to a smaller diameter after a device has passed through central lumen 31. This outer jacket also would serve to couple the reinforcement 38 to the underlying tubular layer 30. In some aspects, the reinforcement 38 can include a series or matrix of holes through the walls of the proximal and distal regions, and an overlying polymer material can be melted through the holes to bond with the underlying tubular layer 30.

As shown in FIG. 6, the reinforcement 38 is arc-shaped in a selected transverse cross section taken perpendicular to a longitudinal axis 36 of the sheath 22. This preserves the ability of sheath 22 to expand in diameter to facilitate the passage of a medical device. The aspect shown has the reinforcement 38 extending around about 50% of the circumference of tubular layer 30. In other aspects, reinforcement 38 might extend around anywhere from 25% of the circumference to about 60% of the circumference of the tubular layer 30 (including about 25%, about 30%, about 35%, about 40%, about 45%, about 50%, about 55%, and about 60%). The reinforcement 38 can extend longitudinally over the full length of the tubular layer 30, or it can extend longitudinally over just a portion of the full length of the tubular layer 30. In some aspects, the reinforcement 38 can extend, at least partially, into or over the material of the tapered proximal region 24. In some aspects, the reinforcement 38 is formed of a rigid polymer. In some aspects, the reinforcement 38 is formed of a metal, such as, but not limited to, stainless steel or nitinol.

Note that the mechanism of expansion of the sheath 22 shown in FIG. 6 is by a longitudinally extending fold 44. Reinforcement 38 is placed opposite to longitudinally extending fold 44 to ensure that the expansion of the sheath is not hindered by the presence of reinforcement 38. Regardless of the expansion mechanism used to allow the sheath to open, the reinforcement will be positioned so as not to interfere with it.

Note that sheath depicted in FIG. 6 also includes a lubricious inner liner 46 positioned radially inward of the tubular layer 30. However, the transaxillary access concepts disclosed herein can be applied to other types of sheaths, and the disclosure is not intended to be limited to this particular sheath aspect.

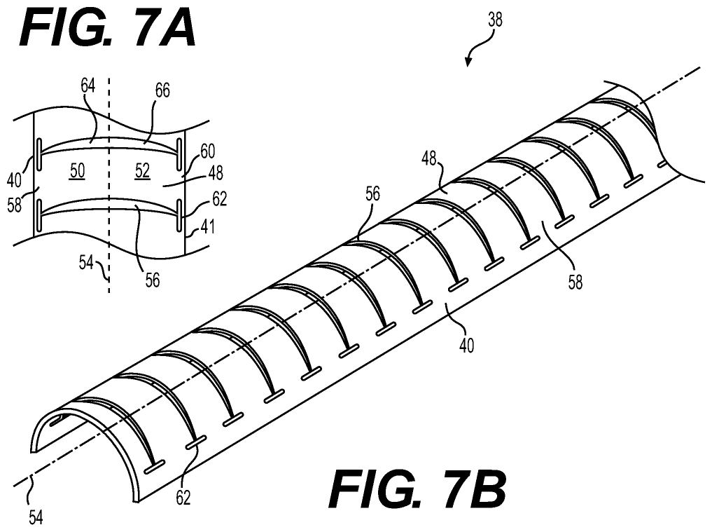

As mentioned above, the reinforcement 38 is biased to bend in an inferior direction under axial compression. In the aspect shown, the bending mechanism is a longitudinally spaced series of ribs 48 separated by circumferentially extending gaps 56 as shown in FIG. 7A and FIG. 7B. The ribs 48 and gaps 56 are positioned between the left and right longitudinally extending spines 40 and 41, which extend uninterrupted the full length of reinforcement 38. Referring now to FIG. 7A, a longitudinally extending rib 48 includes a left intermediate region 50 and a right intermediate region 52, each of which widen while extending circumferentially away from central axis 54 of reinforcement 38. Central axis 54 of extends parallel to the longitudinal axis 36 of the sheath 22. Each rib 48 is spaced from a longitudinally adjacent rib by a circumferentially extending gap 56. The left end of a rib 48 has a left extension 58 that is coupled to a left longitudinally extending spine 40, and the right end of rib 48 has a right extension 60 that is coupled to a right longitudinally extending spine 41.

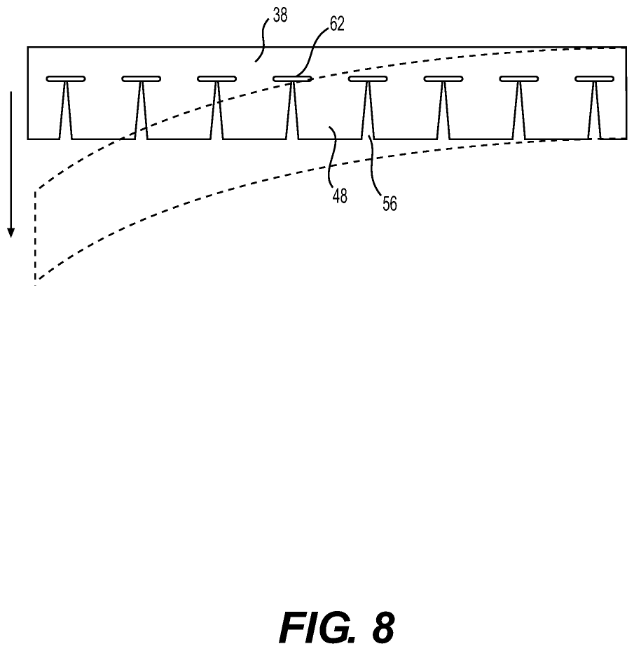

One or more of the gaps of the series of longitudinally spaced gaps, such as gap 56 of FIG. 7A, has an ovoid or elliptical shape. That is, the left side 64 and right side 66 of the ovoid gap 56 each taper, narrowing as they extend circumferentially toward left and right ends of the gap 56. The left and right ends of a gap are wider in the longitudinal direction than the narrowing intermediate portions, forming strain relief regions 62. Strain relief regions 62 are bounded in the longitudinal direction by the extensions 58, 60 of ribs 48. The overall shape of the gaps facilitates bending. As shown in FIG. 8, the reinforcement 38 can bend, or flex, in the inferior direction. The reinforcement 38 bends along left and right longitudinally extending spines 40, 41 as the gaps 56 narrow and the ribs 48 eventually touch. The number and shape of the gaps 56 and the ribs 48, their lengths and their widths, the distance between them, are all variables that can be optimized to obtain a desired flex profile. The bending mechanism allows sheath 22 to bend from a straight configuration to a bent configuration without encountering the kinking problem seen with conventional sheath 8 of FIG. 3. A medical device can pass through the sheath 22 in both a straight configuration and a bent configuration without being significantly obstructed by the walls of sheath 22. In some aspects, the sheath 22 is configured to bend up to 120 degrees without kinking (including for example, bending up to 40 degrees without kinking, bending up to 50 degrees without kinking, bending up to 60 degrees without kinking, bending up to 70 degrees without kinking, bending up to 80 degrees without kinking, bending up to 90 degrees without kinking, bending up to 100 degrees without kinking, bending up to 110 degrees without kinking, and bending up to 120 degrees without kinking).

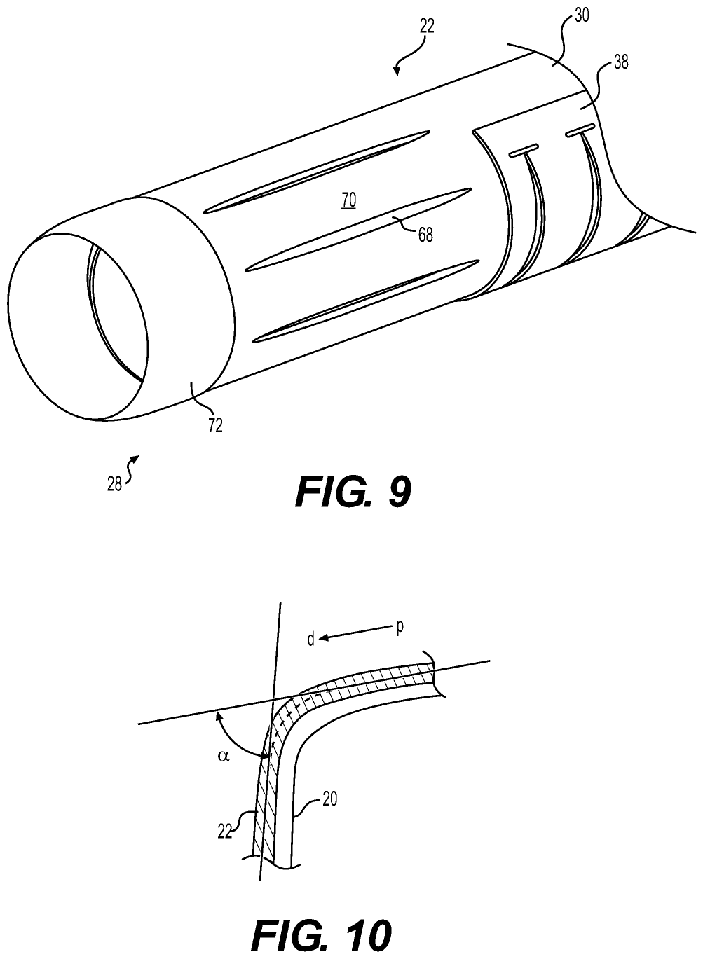

The distal region 28 of the sheath 22 is adapted to be especially atraumatic so as not to injure the subclavian artery as it travels toward the aortic arch. FIG. 9 shows close up view of distal region 28. The distal region 28 includes a plurality of longitudinal slits 68 extending through the wall of tubular layer 30. The slits 68 divide the tubular layer 30 into a plurality of struts 70 that are arranged circumferentially around distal region 28. The slits 68 and struts 70 are positioned distal to reinforcement 38. Upon axial compression, such as might be applied when the distal region 28 encounters a bend in an artery or contacts the native aortic valve, the distal region 28 transforms, taking a bowed configuration wherein each elongated slit 68 widens circumferentially and each strut of plurality of struts 70 bows radially away from the longitudinal axis 36 of sheath 22. The bowed configuration of the distal region 28 is shorter in the longitudinal direction than the unbowed configuration of the distal region 28. The shorter bowed configuration functions to reduce the force to deflect the distal region 28, thereby exerting a lower force on the vasculature, reducing the risk of damage. The distal region 28 can also include a distal tip 72 formed of a soft and flexible material having a lower durometer than the material of the tubular layer 30. The number and shape of the slits 68 and the struts 70, their lengths and their widths, and the distance between them are all variables that can be optimized to obtain the desired level of distal region collapse.

It will be understood that some aspects of a sheath 22 for transaxillary access can include an atraumatic distal region 28 with struts 70 and slits 68 without also including a reinforcement 38. Likewise, some aspects of a sheath 22 for transaxillary access can include a reinforcement 38 without also including an atraumatic distal region 28 with struts 70 and slits 68.

The sheaths 22 disclosed herein can be fabricated by forming a tubular layer 30 to expand when an inner surface of the tubular layer 30 is subjected to a radially outwardly directed force. Some methods of fabrication will include biasing a reinforcement 38 to bend to a greater degree in a first direction than in a second, opposite direction, and coupling the reinforcement to an inferior longitudinal region 34 of the tubular layer 30 to make the sheath 22. Some methods of fabrication will include altering a distal region 28 of the tubular layer to be transformable to a bowed configuration under axial compression. The tubular layer 30 is formed to be from about 4 inches to about 10 inches in length, and includes distal region 28, central shaft 26, and a tapered proximal region 24 that widens in diameter toward a proximal end of the tubular layer 30.

In some methods of fabrication, coupling the reinforcement 38 to the tubular layer 30 can include applying an adhesive to the inferior longitudinal region 34 of the tubular layer 30 and compressing the reinforcement 38 to the adhesive. In some aspects, coupling the reinforcement 38 to the tubular layer 30 can include layering a polymer material over a proximal portion of the reinforcement 38 and layering the polymer material over the distal portion of the reinforcement 38. In some methods of fabrication, the polymer material may be melted, reflowed, and/or heat bonded to the reinforcement 38 and to the underlying tubular layer 30. For example, the polymer material, which may be the same or a different material than the tubular layer 30, can extend longitudinally past the ends of the reinforcement to also contact the tubular layer 30. Or, in some aspects, the reinforcement 38 can include a series or matrix of holes through the walls of the proximal and distal regions, and the polymer material can be melted through the holes to bond with the underlying tubular layer 30. In some methods of fabrication, coupling the reinforcement 38 to the tubular layer 30 includes positioning an outer jacket 42 over and around the tubular layer 30 and reinforcement 38.

The methods of fabrication can further include shaping the reinforcement 38 to have an arc-shape in a transverse cross section. In some methods, the reinforcement 38 can be cut from a flat sheet of material having a higher durometer than tubular layer 30, then bent around a mandrel, under heat, for example, to achieve the arc shape. In some aspects, the reinforcement 38 could be bent around pre-formed tubular layer 30 in order to promote a good fit around the tubular layer 30. In some methods, the reinforcement 38 can cut out of a preformed rigid tube. The rigid tube has a durometer higher than the durometer of the tubular layer 30. The process of biasing the reinforcement 38 can include cutting out a series of longitudinally spaced gaps 56. In some methods of fabrication, one or more gaps 56 can be cut to include left and right sides 64, 66 that narrow extending circumferentially away from a central axis 54 of the reinforcement 38. In some methods, one or more gaps 56 can be cut to include strain relief regions 62 that widen the length of the at least one gap at left and right ends of the gap.

As noted above, some methods of fabrication will include altering a distal region 28 of the tubular layer 30 to be transformable to a bowed configuration under axial compression. For example, a plurality of elongated slits 68 can be cut through the wall thickness of distal region 28 at a position distal to the reinforcement 38 using, for example, a laser or a sharpened tool. Alternatively, the tubular layer 30 can be coextruded with a solid sacrificial material in the position of the slits, which can then be removed to create the slits 68. The slits 68 are circumferentially arranged around the distal region 28, forming circumferentially spaced struts 70. Axial force applied to distal tip 72 causes struts 70 to bow radially outward. Distal tip 72 can be formed of a lower durometer material than the distal region 28, and it can be coupled to distal region 28 of tubular layer 30 using adhesives, heat bonding or coextrusion. In one example, the distal tip 72 could be coextruded with a tie layer beneath it (i.e., the tie layer would be positioned between the material of tubular layer 30 and the material of distal tip 72). The tie layer would serve to more strongly adhere the tubular layer 30 to the material of distal tip 72.

To deliver a medical device to a procedure site, a practitioner inserts an expandable sheath 22 into a vessel (such as, but not limited to, the subclavian artery). The distal tip 72 of the expandable sheath moves toward a bend in the vessel. As the distal tip 72 meets the vessel wall at the bend, the vessel wall exerts a force on the distal tip 72. Distal region 28 of the sheath 22 transforms to an at least partially bowed configuration to absorb force applied to the distal tip 72 by the vessel wall. In some aspects, distal region 28 comprises a plurality of elongated struts 70 circumferentially arranged around the distal region as shown in FIG. 9. Transforming the distal region 28 to an at least partially bowed configuration comprises bowing at least one strut of the plurality of elongated struts 70 in an outward radial direction and widening at least one slit 68 in a circumferential direction. The distal region 28 is shortened when the distal region takes a bowed configuration.

As the distal tip 72 advances past the bend, the central shaft 26 of the sheath is bent as it moves along the bend in the vessel. The central shaft 26 of the sheath 22 can be bent to an angle up to about 120 degrees without kinking (including up to about 30 degrees, up to about 40 degrees, up to about 50 degrees, up to about 60 degrees, up to about 70 degrees, up to about 80 degrees, up to about 90 degrees, up to about 100 degrees, up to about 110 degrees, and up to about 120 degrees without kinking). FIG. 10 depicts a sheath 22 that, moving in the direction of the arrow from a proximal location p toward a distal location d, has navigated a bend in a tortuous vessel 20 without kinking. The sheath has bent to angle a, wherein the luminal axis of the sheath 22 distal to the bend creates angle a with the luminal axis of the sheath 22 proximal to the bend.

When a reinforcement 38 is included, as shown in FIG. 4 and FIG. 8, the reinforcement 38 is biased to bend in one direction under axial compression. When sheath 22 is inserted into the subclavian artery, it is positioned such that the reinforcement 38 is located on the inferior side of the sheath 22. As such, the sheath 22 will bend in the inferior direction when it meets the bend B shown in FIG. 2. Bending the reinforcement 38 results in the bending of left and right longitudinally extending spines 40, 41 and the narrowing of one or more ovoid gaps 56 in a longitudinal direction. As reinforcement 38 bends, proximal and distal edges of gaps 56 begin to come into contact. The reinforcement 38 can bend until the proximal and distal edges of each gap 56 contact with each other. In some aspects, bending strain is relieved via a series of strain relief regions 62, preventing stress cracks from forming or propagating and making it easier to flex the reinforcement 38 by closing the gaps 56.

The outer diameter of the medical device is wider than the inner diameter of the sheath 22, and therefore applies an outwardly directed radial force on the inner surface of the sheath 22. The sheath 22 locally expands at the location of the medical device to accommodate the medical device as it moves through the lumen 31. Once the medical device has advanced past a selected longitudinal position, the sheath 22 locally collapses back to a smaller diameter at the selected position due to the withdrawal of the outwardly directed radial force. For example, some aspects of the sheath include an outer jacket 42. In some aspects, the outer jacket 42 is elastomeric. An elastomeric outer jacket can be stretched to a larger diameter, but has a bias toward its original diameter. When outer jacket 42 is stretched by the passage of a medical device, it applies an inwardly directed radial force on the tubular layer 30 of sheath 22 to cause it to collapse back to a smaller diameter. Finally, the medical device exits the distal tip 72 of the sheath 22 and is delivered to the procedure site. For example, the medical device can be a transcatheter heart valve delivered to the native aortic valve.

### EXAMPLE

This example details an expandable sheath designed for use in a TAVR procedure conducted specifically from the transaxillary access. The example sheath has 3 main features differing from conventional transfemoral sheaths in order to meet the specific requirements of the transaxillary access. The sheath shaft is shorter in length than a transfemoral sheath, such that the sheath can be fully inserted in the transaxillary anatomy and sutured in place while the distal end of the sheath lies just above the aortic valve. A laser-cut hypotube is added axially along the sheath shaft as a reinforcement, lying on only a portion of the radius of the shaft's cross section. The hypotube is positioned on the area of the sheath shaft that will be placed on the inner curvature of the bend from the subclavian artery to the aortic arch in order to prevent kinking of the transaxillary sheath. Finally, as compared to conventional transfemoral sheaths, the distal tip is made of a softer, more flexible material, and relief cuts are made into the distal region in order to lessen the force exerted by the sheath onto the anatomy when contacting the walls of the aorta or native valve. Together, the design improves upon the procedure for a transaxillary TAVR by, at least: (1) allowing the physician to suture the sheath in place, reducing the need for extra staff, (2) preventing kinking of the sheath shaft, thereby mitigating the risk of the high push force required to advance a prosthetic through a collapsed lumen (or total inability to complete the procedure), and (3) reducing damage to the patient vasculature and heart by providing a less traumatic tip.

The main body of the transaxillary sheath shaft is shorter in length than conventional transfemoral sheaths. For example, a transaxillary sheath shaft can be about 7 inches long, which is 7 inches shorter than an exemplary transfemoral sheath, which is about 14 inches.

A laser cut hypotube is added axially to the sheath shaft, along one side of the sheath, between the sheath's outer jacket and inner member. The hypotube is made of metal, such as stainless steel. The hypotube is cut in half to make a semicircle shape. Along the hypotube, thin cuts are made through the material at an angle, creating gaps in the tubing (as shown in FIGS. 7A and 7B). The gaps create corresponding bands of material across the shaft, called ribs. These gaps are spaced at regular intervals along the length of the hypotube. Elliptical cutouts at the back edge of the angled cuts act to relieve strain at the termination of the cuts. The cutouts are made through the shaft and around most of the semicircle. At the edges of the semicircle, two spines are present, where the metal runs uninterrupted for the full length of the hypotube.

The cuts allow the hypotube to bend, or flex, in a single direction, as shown in FIG. 8. At each cut, the hypotube can bend as the cut becomes smaller and smaller, until there is no longer any gap between two consecutive ribs, and they touch. The cuts are made along the length of the hypotube, allowing the entire length of the component to flex. The final flex profile is determined when the hypotube bends enough so that all ribs are touching, and there are no longer any gaps in the tubing whatsoever. The example shown here is modelled with arbitrary geometry, but on the final hypotube, the dimensions of the cuts are designed such that the final flex profile of the hybotube is able to bend at a 90 degree angle to approximate the mean transition from the subclavian artery to the aortic arch. This hypotube would flex sufficiently to allow for easy tracking from the left subclavian artery into the aortic arch.

The hyptotube is made to a radius just larger than that of the unexpanded outer diameter of the sheath inner member and placed axially along the sheath shaft. The length of the hyptotube extends from the end of the proximal taper section, to just proximal to the distal tip. The hypotube is bonded to the sheath inner member along the length of the hyptotube's spine, to hold it securely around the shaft.

The hyptotube is placed in between the inner member and the outer jacket, so it interacts with neither interventional devices within the sheath, nor the patient anatomy outside the sheath. Radially, the hypotube is positioned across from the expanding portion of the sheath. For example, in FIG. 6, the inner member of the expanding section unfolds with the passage of the valve, while the outer jacket stretches. Opposite the expanding portion, where the hypotube is located, the inner member and outer jacket do not move or stretch during expansion. Thus, the hypotube does not affect the ability of the sheath to expand, and the force to advance interventional devices through the sheath is not impacted.

The hypotube is passively flexed. Instead of using pull wires, the hypotube naturally bends with the anatomy as the sheath tracks over the guidewire. Conventional transfemoral sheaths kink when inserted through a sufficiently small bend. Kinking is a partial or full collapse of the inner lumen on the sheath shaft. The kinking typically originates in the more rigid layers of the transfemoral sheaths. The sheath kinks along the inner curvature of a bend. The collapse of the inner lumen causes difficulty or may fully prevent the passage of interventional devices through the sheath. In this example transaxillary sheath, the hypotube is oriented so that it lies on the inner curve of the bend from the subclavian artery into the aortic arch. When tracking through this area, the hypotube will conform to the curvature of the anatomy. In doing so, the hypotube supports the sheath shaft on the inner curvature, preventing the wall of the inner member from collapsing inward, stopping kinking from occurring.

The distal tip of the sheath is made up a softer, more flexible material than conventional transfemoral sheaths. For example instead of linear low-density polyethylene (LLDPE), the example transaxillary sheath tip can be formed of rubber. LLDPE tips are harder and less flexible. They may also have sharp edges and corners after they tear to allow the valve to pass through. A softer rubber material is more ideal for transaxillary access, when interaction with the native anatomy is a possibility.

Additionally, the distal region of the sheath shaft contains thin slits, or relief cuts, radially patterned around the circumference (see FIG. 9). The cuts extend through the inner member surface. When the tip is pressed by the vessel wall, the relief cuts allow the wall to extend radially outward, shortening the shaft. This diffuses the force outwards and lessens the force that the sheath tip can exert on the anatomy, making it less traumatic. The exact dimensions and number of instances of the relief cuts can be varied to produce the desired performance. The hypotube terminates just proximal to the relief cuts, as the very distal edge of the sheath shaft does not need to bend.

### Exemplary Aspects

In view of the described processes and compositions, hereinbelow are described certain more particularly described aspects of the disclosures. These particularly recited aspects should not, however, be interpreted to have any limiting effect on any different claims containing different or more general teachings described herein, or that the “particular” aspects are somehow limited in some way other than the inherent meanings of the language and formulas literally used therein.

Example 1: An expandable sheath comprising: a central lumen and a longitudinal axis extending therethrough; a tubular layer comprising an inferior longitudinal region and a superior longitudinal region; and a longitudinally extending reinforcement coupled to the inferior longitudinal region and biased to bend in an inferior direction under axial compression, the reinforcement comprising a central axis extending parallel to the longitudinal axis of the sheath; wherein the sheath is expandable from a collapsed configuration to a radially expanded configuration to facilitate passage of a medical device through the central lumen of the sheath.

Example 2: The expandable sheath according to any example herein, particularly example 1, wherein the tubular layer further comprises a tapered proximal region, a distal region, and a central shaft extending distally therebetween.

Example 3: The expandable sheath according to any example herein, particularly examples 1-2, wherein a length of the sheath is from 4 inches to 10 inches.

Example 4: The expandable sheath according to any example herein, particularly examples 1-3, wherein a durometer of the reinforcement is higher than a durometer of the tubular layer.

Example 5: The expandable sheath according to any example herein, particularly examples 1-4, wherein the reinforcement is coupled to an external surface of the inferior longitudinal region.

Example 6: The expandable sheath according to any example herein, particularly example 5, further comprising an adhesive coupling the inferior longitudinal region to a longitudinally extending spine extending along a longitudinal edge of the reinforcement.

Example 7: The expandable sheath according to any example herein, particularly examples 5-6, further comprising a layer of polymer positioned over a proximal portion of the reinforcement and a distal portion of the reinforcement.

Example 8: The expandable sheath according to any example herein, particularly example 7, wherein the layer of polymer does not cover a longitudinally central region of the reinforcement.

Example 9: The expandable sheath according to any example herein, particularly examples 7-8, wherein the tubular layer and the layer of polymer are formed of the same material.

Example 10: The expandable sheath according to any example herein, particularly examples 5-9, wherein an outer jacket extends over the reinforcement and the tubular layer.

Example 11: The expandable sheath according to any example herein, particularly examples 5-10, wherein the superior longitudinal region of the tubular layer further comprises at least one longitudinally extending fold.

Example 12: The expandable sheath according to any example herein, particularly examples 1-11, wherein the reinforcement is arc-shaped at a selected transverse cross section taken perpendicular to the longitudinal axis of the sheath.

Example 13: The expandable sheath according to any example herein, particularly example 12, wherein at the selected transverse cross section, the reinforcement extends around from 25% of a circumference of the tubular layer to 60% of the circumference of the tubular layer.

Example 14: The expandable sheath according to any example herein, particularly examples 1-13, wherein the reinforcement comprises a series of longitudinally spaced ribs, each rib comprising left and right intermediate regions extending circumferentially away the central axis of the reinforcement.

Example 15: The expandable sheath according to any example herein, particularly example 14, wherein each rib of the series of longitudinally spaced ribs is spaced from a longitudinally adjacent rib of the series of longitudinally spaced ribs by a circumferentially extending gap.

Example 16: The expandable sheath according to any example herein, particularly examples 14-15, wherein each rib of the series of longitudinally spaced ribs is coupled at a left end to a left longitudinally extending spine, and each rib of the series of longitudinally spaced ribs is coupled at a right end to a right longitudinally extending spine.

Example 17: The expandable sheath according to any example herein, particularly example 16, wherein a selected rib of the series of longitudinally spaced ribs comprises a left extension extending from the left intermediate region and a right extension extending from the right intermediate region, the left extension being coupled to the left longitudinally extending spine and the right extension being coupled to the right longitudinally extending spine.

Example 18: The expandable sheath according to any example herein, particularly examples 16-17, wherein a material of the left longitudinally extending spine extends uninterrupted a full length of the reinforcement and a material of the right longitudinally extending spine extends uninterrupted the full length of the reinforcement.

Example 19: The expandable sheath according to any example herein, particularly examples 14-18, wherein the intermediate regions of a selected rib widen extending away from the central axis of the reinforcement.

Example 20: The expandable sheath according to any example herein, particularly examples 14-19, wherein the series of longitudinally spaced ribs define a series of longitudinally spaced gaps, each gap comprising a left side extending circumferentially away from the central axis and a right side extending circumferentially away from the central axis.

Example 21: The expandable sheath according to any example herein, particularly example 20, wherein the series of longitudinally spaced gaps is bounded on a left end by the left longitudinally extending spine, and the series of longitudinally spaced gaps is bounded on a right end by the right longitudinally extending spine.

Example 22: The expandable sheath according to any example herein, particularly examples 20-21, wherein the left side of a selected gap comprises a left taper that narrows approaching the left end of the selected gap, and the right side of the selected gap comprises a right taper that narrows approaching the right end of the selected gap.

Example 23: The expandable sheath according to any example herein, particularly example 22, wherein the left side of a selected gap comprises a widening strain relief portion between the left taper and the left end of the selected gap, and the right side of the selected gap comprises a widening strain relief portion between the right taper and the right end of the selected gap.

Example 24: The expandable sheath according to any example herein, particularly examples 14-23, wherein the series of longitudinally spaced ribs define a series of longitudinally spaced gaps, and a selected gap of the series of longitudinally spaced gaps has an ovoid shape narrowing circumferentially toward left and right ends then widening to terminate in strain relief regions at the left and right ends.

Example 25: The expandable sheath according to any example herein, particularly example 24, wherein the strain relief regions are elongated in a longitudinal direction.

Example 26: The expandable sheath according to any example herein, particularly example 25, wherein a selected strain relief region is partially defined by an extension of an adjacent rib.

Example 27: The expandable sheath according to any example herein, particularly examples 1-26, wherein the tubular layer further comprises a distal region, the distal region comprising a plurality of struts, the plurality of struts at least partially defining a plurality of elongated slits extending through a wall thickness of the distal region.

Example 28: The expandable sheath according to any example herein, particularly example 27, wherein the plurality of struts is arranged circumferentially around the distal region.

Example 29: The expandable sheath according to any example herein, particularly examples 27-28, wherein the plurality of struts is positioned distal to the reinforcement.

Example 30: The expandable sheath according to any example herein, particularly examples 27-29, wherein upon axial compression the distal region transforms to a bowed configuration wherein each elongated slit widens circumferentially and each strut bows radially away from the longitudinal axis, thereby shortening the distal region.

Example 31: The expandable sheath according to any example herein, particularly examples 27-30, wherein the distal region is coupled to a distal tip, and the distal tip is formed of a lower durometer material than a material of the distal region.

Example 32: The expandable sheath according to any example herein, particularly examples 27-31, wherein the distal region further comprises at least one longitudinally extending fold extending along a portion of the central shaft of the tubular layer and a portion of the distal region of the tubular layer.

Example 33: The expandable sheath according to any example herein, particularly examples 1-32, wherein the sheath is configured to bend without kinking.

Example 34: The expandable sheath according to any example herein, particularly example 33, wherein the sheath is bendable from a straight configuration to a bent configuration, both the straight configuration and the bent configuration facilitating unobstructed passage of the medical device through the central lumen of the sheath.

Example 35: An expandable sheath comprising: a central lumen and a longitudinal axis extending therethrough; a tubular layer comprising a central shaft and a distal region, the distal region transformable to a bowed configuration under axial compression, the bowed configuration of the distal region comprising a larger diameter than the shaft of the tubular layer;

and a distal tip, wherein the distal tip is formed of a lower durometer material than a material of the tubular layer; wherein the sheath is expandable from a collapsed configuration to a radially expanded configuration to facilitate passage of a medical device through the central lumen of the sheath.

Example 36: The expandable sheath according to any example herein, particularly example 35, wherein the tubular layer further comprises a tapered proximal region.

Example 37: The expandable sheath according to any example herein, particularly examples 35-36, wherein a length of the sheath is from 4 inches to 10 inches.

Example 38: The expandable sheath according to any example herein, particularly examples 35-37, wherein a reinforcement is coupled to an external surface of an inferior longitudinal region of the tubular layer.

Example 39: The expandable sheath according to any example herein, particularly example 38, further comprising an adhesive coupling the inferior longitudinal region to a longitudinally extending spine extending along a longitudinal edge of the reinforcement.

Example 40: The expandable sheath according to any example herein, particularly examples 38-39, further comprising a layer of polymer positioned over a proximal portion of the reinforcement and a distal portion of the reinforcement.

Example 41: The expandable sheath according to any example herein, particularly example 40, wherein the layer of polymer does not cover a longitudinally central region of the reinforcement.

Example 42: The expandable sheath according to any example herein, particularly examples 40-41, wherein the tubular layer and the layer of polymer are formed of a same material.

Example 43: The expandable sheath according to any example herein, particularly examples 38-42, wherein an outer jacket extends over the reinforcement and the tubular layer.

Example 44: The expandable sheath according to any example herein, particularly examples 38-43, wherein a superior longitudinal region of the tubular layer further comprises at least one longitudinally extending fold.

Example 45: The expandable sheath according to any example herein, particularly examples 38-44, wherein the reinforcement is arc-shaped in a selected transverse cross section taken perpendicular to the longitudinal axis of the sheath.

Example 46: The expandable sheath according to any example herein, particularly examples 35-46, wherein the reinforcement comprises a series of longitudinally spaced ribs, each rib comprising a left intermediate region extending circumferentially away from a central axis of the reinforcement and a right intermediate region extending circumferentially away from the central axis of the reinforcement.

Example 47: The expandable sheath according to any example herein, particularly example 46, wherein each rib of the series of longitudinally spaced ribs is spaced from a longitudinally adjacent rib of the series of longitudinally spaced ribs by a circumferentially extending gap.

Example 48: The expandable sheath according to any example herein, particularly examples 46-47, wherein each rib of the series of longitudinally spaced ribs is coupled at a left end to a left longitudinally extending spine and each rib of the series of longitudinally spaced ribs is coupled at a right end to a right longitudinally extending spine.

Example 49: The expandable sheath according to any example herein, particularly example 48, wherein a selected rib of the series of longitudinally spaced ribs comprises a left extension extending from the left intermediate region and a right extension extending from the right intermediate region, the left extension being coupled to the left longitudinally extending spine and the right extension being coupled to the right longitudinally extending spine.

Example 50: The expandable sheath according to any example herein, particularly examples 48-49, wherein a material of the left longitudinally extending spine extends uninterrupted a full length of the reinforcement and a material of the right longitudinally extending spine extends uninterrupted the full length of the reinforcement.

Example 51: The expandable sheath according to any example herein, particularly examples 46-50, wherein the intermediate regions of a selected rib widen extending away from the central axis of the reinforcement.

Example 52: The expandable sheath according to any example herein, particularly examples 46-51, wherein the series of longitudinally spaced ribs define a series of longitudinally spaced gaps, each gap comprising a left side extending circumferentially away from the central axis and a right side extending circumferentially away from the central axis.

Example 53: The expandable sheath according to any example herein, particularly example 52, wherein the series of longitudinally spaced gaps is bounded on a left end by the left longitudinally extending spine, and the series of longitudinally spaced gaps is bounded on a right end by the right longitudinally extending spine.

Example 54: The expandable sheath according to any example herein, particularly examples 52-53, wherein the left side of a selected gap comprises a left taper that narrows approaching the left end of the selected gap, and the right side of the selected gap comprises a right taper that narrows approaching the right end of the selected gap.

Example 55: The expandable sheath according to any example herein, particularly examples 54, wherein the left side of a selected gap comprises a widening strain relief portion between the left taper and the left end of the selected gap, and the side region of the selected gap comprises a widening strain relief portion between the right taper and the right end of the selected gap.

Example 56: The expandable sheath according to any example herein, particularly examples 46-56, wherein the series of longitudinally spaced ribs define a series of longitudinally spaced gaps, and a selected gap of the series of longitudinally spaced gaps has an ovoid shape narrowing circumferentially toward left and right ends then widening to terminate in strain relief regions at the left and right ends.

Example 57: The expandable sheath according to any example herein, particularly example claim 56, wherein the strain relief regions are elongated in a longitudinal direction.

Example 58: The expandable sheath according to any example herein, particularly example claim 57, wherein a selected strain relief region is partially defined by an extension of an adjacent rib.

Example 59: The expandable sheath according to any example herein, particularly examples 35-58, wherein the distal region of the tubular layer comprises a plurality of struts, the plurality of struts at least partially defining a plurality of elongated slits extending through a wall thickness of the distal region, wherein under axial compression each elongated slit widens circumferentially and each strut bows radially away from the longitudinal axis taking the bowed configuration.

Example 60: The expandable sheath according to any example herein, particularly example 59, wherein the plurality of struts is arranged circumferentially around the distal region of the tubular layer.

Example 61: The expandable sheath according to any example herein, particularly examples 59-60, wherein the distal region further comprises at least one longitudinally extending fold extending along a portion of the central shaft of the tubular layer and a portion of the distal region of the tubular layer.

Example 62: The expandable sheath according to any example herein, particularly examples 59-61, wherein the plurality of struts is positioned distal to the reinforcement.

Example 63: The expandable sheath according to any example herein, particularly examples 59-62, wherein a length of the distal region is shorter in the bowed configuration than in an unbowed configuration.

Example 64: The expandable sheath according to any example herein, particularly examples 35-63, wherein the sheath is configured to bend without kinking.

Example 65: The expandable sheath according to any example herein, particularly example 64, wherein the sheath is bendable from a straight configuration to a bent configuration, both the straight configuration and the bent configuration facilitating unobstructed passage of the medical device through the central lumen of the sheath.

Example 66: A method of making an expandable sheath, the method comprising; forming a tubular layer to expand when an inner surface of the tubular layer is subjected to a radially outwardly directed force; biasing a reinforcement to bend to a greater degree in a first direction than in the second, opposite direction; and coupling the reinforcement to an inferior longitudinal region of the tubular layer to make the expandable sheath.

Example 67: The method according to any example herein, particularly example 66, wherein forming the tubular layer comprises forming a tapered proximal region that widens toward a proximal end of the tubular layer.

Example 68: The method according to any example herein, particularly examples 66-67, wherein a length of the tubular layer is from 4 inches to 10 inches.

Example 69: The method according to any example herein, particularly examples 66-68, wherein coupling the reinforcement comprises applying an adhesive to the inferior longitudinal region of the tubular layer.

Example 70: The method according to any example herein, particularly example 69, further comprising compressing a longitudinally extending spine of the reinforcement to the adhesive.

Example 71: The method according to any example herein, particularly examples 66-70, wherein coupling the reinforcement comprises layering a polymer material over a proximal portion of the reinforcement and layering the polymer material over a distal portion of the reinforcement.

Example 72: The method according to any example herein, particularly example 71, wherein layering the polymer material over proximal and distal portions of the reinforcement comprise melting the polymer material and applying the melted polymer material to the reinforcement.

Example 73: The method according to any example herein, particularly example 72, wherein the polymer material is formed of a same material as the tubular layer.

Example 74: The method according to any example herein, particularly examples 66-73, wherein coupling the reinforcement comprises positioning an outer jacket over the reinforcement and the tubular layer.

Example 75: The method according to any example herein, particularly examples 66-74, further comprising shaping the reinforcement to have an arc-shape in a transverse cross section.

Example 76: The method according to any example herein, particularly example 75, wherein shaping the reinforcement comprises cutting the reinforcement out of a rigid tube, wherein the rigid tube has a durometer higher than the durometer of the tubular layer.

Example 77: The method according to any example herein, particularly examples 66-76, wherein biasing the reinforcement comprises cutting out a series of longitudinally spaced gaps.

Example 78: The method according to any example herein, particularly example 77, wherein cutting out the series of longitudinally spaced gaps further comprises cutting out at least one gap to include left and right sides that each narrow extending circumferentially away from a central axis of the reinforcement.

Example 79: The method according to any example herein, particularly examples 77-78, wherein cutting out the series of longitudinally spaced gaps further comprises cutting out at least one gap to include left and right strain relief regions that widen a length of the at least one gap at left and right ends of the gap.

Example 80: The method according to any example herein, particularly examples 66-79, further comprising altering a distal region of the tubular layer to be transformable to a bowed configuration under axial compression.

Example 81: The method according to any example herein, particularly example 80, further comprising coupling a distal tip to the distal region of the tubular layer, the distal tip being formed of a lower durometer material than the distal region.

Example 82: The method according to any example herein, particularly examples 80- 81, wherein altering the distal region of the tubular layer comprises cutting a plurality of elongated slits through a wall thickness of the distal region of the tubular layer, wherein the plurality of elongated slits is arranged circumferentially around the distal region of the tubular layer.

Example 83: The method according to any example herein, particularly examples 80-82, wherein the reinforcement is coupled proximal to the plurality of elongated slits.

Example 84: A method of making an expandable sheath, the method comprising; forming a tubular layer to expand when an inner surface of the tubular layer is subjected to a radially outwardly directed force; altering a distal region of the tubular layer to be transformable to a bowed configuration under axial compression; and coupling a distal tip to the distal region of the tubular layer to make the expandable sheath, the distal tip being formed of a lower durometer material than the distal region.

Example 85: The method according to any example herein, particularly example 84, wherein forming the tubular layer comprises forming a tapered proximal region that widens toward a proximal end of the tubular layer.

Example 86: The method according to any example herein, particularly examples 84-85, wherein a length of the tubular layer is from 4 inches to 10 inches.

Example 87: The method according to any example herein, particularly examples 84-86, further comprising biasing a reinforcement to bend to a greater degree in a first direction than in a second, opposite direction and coupling the reinforcement to an inferior longitudinal region of the tubular layer.

Example 88: The method according to any example herein, particularly example 87, wherein coupling the reinforcement comprises applying an adhesive to the inferior longitudinal region of the tubular layer.

Example 89: The method according to any example herein, particularly example 88, further comprising compressing a longitudinally extending spine of the reinforcement to the adhesive.

Example 90: The method according to any example herein, particularly examples 87-89, wherein coupling the reinforcement comprises layering a polymer material over a proximal portion of the reinforcement and layering the polymer material over a distal portion of the reinforcement.

Example 91: The method according to any example herein, particularly example 90, wherein layering the polymer material over the proximal and distal portions of the reinforcement comprises melting the polymer material and applying the melted polymer material to the reinforcement.

Example 92: The method according to any example herein, particularly example 91, wherein the polymer material is formed of a same material as the tubular layer.

Example 93: The method according to any example herein, particularly examples 87-92, wherein coupling the reinforcement comprises positioning an outer jacket over the reinforcement and the tubular layer.

Example 94: The method according to any example herein, particularly examples 87-93, further comprising shaping the reinforcement to have an arc-shape in a transverse cross section.

Example 95: The method according to any example herein, particularly example 94, wherein shaping the reinforcement comprises cutting the reinforcement out of a rigid tube, wherein the rigid tube has a durometer higher than the durometer of the tubular layer.

Example 96: The method according to any example herein, particularly examples 87-95, wherein biasing the reinforcement comprises cutting out a series of longitudinally spaced gaps.

Example 97: The method according to any example herein, particularly example 96, wherein cutting out the series of longitudinally spaced gaps further comprises cutting out at least one gap to include left and right strain relief regions that widen a length of the at least one gap at left and right ends of the gap.

Example 98: The method according to any example herein, particularly examples 96-97, wherein cutting out a series of longitudinally spaced gaps further comprises cutting out at least one gap to include left and right strain relief regions that widen the length of the at least one gap at left and right ends of the gap.

Example 99: The method according to any example herein, particularly examples 84-98, wherein altering the distal region of the tubular layer comprises cutting a plurality of elongated slits through a wall thickness of the distal region, wherein the plurality of elongated slits is arranged circumferentially around the distal region of the tubular layer.

Example 100: The method according to any example herein, particularly example 99, further comprising coupling a reinforcement to the tubular layer proximal to the plurality of elongated slits.

Example 101: A method of delivering a medical device to a procedure site, the method comprising; inserting an expandable sheath into a vessel of a subject; moving a distal tip of the expandable sheath toward a bend in the vessel; exerting pressure on the distal tip of the expandable sheath via contact with a vessel wall at the bend; transforming a distal region of the expandable sheath to an at least partially bowed configuration; advancing the distal tip of the expandable sheath distally past the bend in the vessel; bending a central shaft of the expandable sheath to an angle up to 120 degrees as the central shaft moves through the bend in the vessel; advancing the medical device through a central lumen of the expandable sheath; locally expanding the expandable sheath at a selected longitudinal position via an outwardly directed radial force applied by the advancing medical device; advancing the medical device through a bent portion of the central shaft; locally collapsing the expandable sheath at the selected longitudinal position once the medical device has advanced to a more distal location; and delivering the medical device to the procedure site.

Example 102: The method according to any example herein, particularly example 101, wherein the vessel is a subclavian artery.

Example 103: The method according to any example herein, particularly examples 101-102, wherein the procedure site is a aortic valve.

Example 104: The method according to any example herein, particularly examples 101-103, wherein the medical device advances unobstructed through the bent portion of the central shaft.

Example 105: The method according to any example herein, particularly examples 101-104, further comprising contacting arterial surfaces with an outer jacket material.

Example 106: The method according to any example herein, particularly example 105, wherein locally collapsing the expandable sheath comprises applying an inwardly directed radial force on a tubular layer of the sheath via an inward bias of the outer jacket material.

Example 107: The method according to any example herein, particularly examples 101-106, wherein the distal region of the tubular layer further comprises a plurality of elongated struts circumferentially arranged around the distal region of the tubular layer, and transforming the distal region of the expandable sheath to the at least partially bowed configuration comprises bowing at least one strut of the plurality of elongated struts in an outward radial direction.

Example 108: The method according to any example herein, particularly example 107, wherein the distal region of the tubular layer further comprises a plurality of elongated slits circumferentially arranged around the distal region of the tubular layer, and transforming a distal region of the expandable sheath to the at least partially bowed configuration comprises widening at least one slit of the plurality of elongated slits in a circumferential direction.

Example 109: The method according to any example herein, particularly example 108, wherein transforming a distal region of the expandable sheath to an at least partially bowed configuration comprises shortening the distal region.

Example 110: The method according to any example herein, particularly examples 101-109, wherein bending the central shaft comprises bending a reinforcement that extends longitudinally down one side of the expandable sheath.

Example 111: The method according to any example herein, particularly example 110, wherein the reinforcement comprises a series of ovoid gaps spaced longitudinally along the reinforcement, and bending the reinforcement comprises narrowing one or more ovoid gaps in a longitudinal direction.

Example 112: The method according to any example herein, particularly example 111, wherein bending the reinforcement further comprises relieving strain of the bend via a series of strain relief regions that widen the left and right edges of each ovoid gap.

Example 113: The method according to any example herein, particularly examples 111-112, wherein bending the reinforcement further comprises bending left and right longitudinally extending spines that bound the series of ovoid gaps.

Example 114: The method according to any example herein, particularly examples 111-113, wherein narrowing one or more ovoid gaps further comprises bringing a proximal edge of at least one selected gap into contact with a distal edge of the selected gap.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the form disclosed. Many modifications and variations will be apparent to those of ordinary skill in the art without departing from the scope and spirit of the disclosure. The implementations were chosen and described in order to best explain the principles of the disclosure and the practical application, and to enable others of ordinary skill in the art to understand the invention for various implementations with various modifications as are suited to the particular use contemplated.

### REFERENCES

Biasco, Luigi, et al. “Access Sites for TAVI: Patient Selection Criteria, Technical Aspects, and Outcomes”

What is claimed is:

1. An expandable sheath comprising:
a central lumen and a longitudinal axis extending therethrough;
a tubular layer comprising an inferior longitudinal region and a superior longitudinal region; and
a longitudinally extending reinforcement coupled to the inferior longitudinal region and biased to bend in an inferior direction under axial compression, the reinforcement comprising a central axis extending parallel to the longitudinal axis of the sheath;
wherein the sheath is expandable from a collapsed configuration to a radially expanded configuration to facilitate passage of a medical device through the central lumen of the sheath.

2. The expandable sheath of claim 1, wherein the tubular layer further comprises a tapered proximal region, a distal region, and a central shaft extending distally therebetween.

3. The expandable sheath of claim 1, wherein a length of the sheath is from 4 inches to 10 inches.

4. The expandable sheath of claim 1, wherein a durometer of the reinforcement is higher than a durometer of the tubular layer.

5. The expandable sheath of claim 1, wherein the reinforcement is coupled to an external surface of the inferior longitudinal region.

6. The expandable sheath of claim 5, wherein an outer jacket extends over the reinforcement and the tubular layer.

7. The expandable sheath of claim 5, wherein the superior longitudinal region of the tubular layer further comprises at least one longitudinally extending fold.

8. The expandable sheath of claim 1, wherein the reinforcement is arc-shaped at a selected transverse cross section taken perpendicular to the longitudinal axis of the sheath.

9. The expandable sheath of claim 8, wherein at the selected transverse cross section, the reinforcement extends around from 25% of a circumference of the tubular layer to 60% of the circumference of the tubular layer.

10. The expandable sheath of claim 1, wherein the reinforcement comprises a series of longitudinally spaced ribs, each rib comprising left and right intermediate regions extending circumferentially away the central axis of the reinforcement.

11. The expandable sheath of claim 10, wherein each rib of the series of longitudinally spaced ribs is coupled at a left end to a left longitudinally extending spine, and each rib of the series of longitudinally spaced ribs is coupled at a right end to a right longitudinally extending spine.

12. The expandable sheath of claim 11, wherein a selected rib of the series of longitudinally spaced ribs comprises a left extension extending from the left intermediate region and a right extension extending from the right intermediate region, the left extension being coupled to the left longitudinally extending spine and the right extension being coupled to the right longitudinally extending spine.

13. The expandable sheath of claim 10, wherein the intermediate regions of a selected rib widen extending away from the central axis of the reinforcement.

14. The expandable sheath of claim 10, wherein the series of longitudinally spaced ribs define a series of longitudinally spaced gaps, each gap comprising a left side extending circumferentially away from the central axis and a right side extending circumferentially away from the central axis.

15. The expandable sheath of claim 14, wherein the series of longitudinally spaced gaps is bounded on a left end by a left longitudinally extending spine, and the series of longitudinally spaced gaps is bounded on a right end by a right longitudinally extending spine.

16. The expandable sheath of claim 14, wherein the left side of a selected gap comprises a left taper that narrows approaching a left end of the selected gap, and the right side of the selected gap comprises a right taper that narrows approaching a right end of the selected gap.

17. The expandable sheath of claim 16, wherein the left side of a selected gap comprises a widening strain relief portion between the left taper and the left end of the selected gap, and the right side of the selected gap comprises a widening strain relief portion between the right taper and the right end of the selected gap.

18. The expandable sheath of claim 1, wherein the tubular layer further comprises a distal region, the distal region comprising a plurality of longitudinally extending struts, the plurality of longitudinally extending struts at least partially defining a plurality of elongated slits extending through a wall thickness of the distal region.

19. The expandable sheath of claim 18, wherein upon axial compression the distal region transforms to a bowed configuration wherein each elongated slit widens circumferentially and each strut bows radially away from the longitudinal axis, thereby shortening the distal region.

20. The expandable sheath of claim 1, wherein the sheath is bendable from a straight configuration to a bent configuration, both the straight configuration and the bent configuration facilitating unobstructed passage of the medical device through the central lumen of the sheath.
